(19) United States
(12) Patent Application Publication
(10) Pub. No.: US 2006/0014219 A1
(43) Pub. Date: Jan. 19, 2006

(54) Method for measuring ion channel activity

(76) Inventors: Ruihua Chen, Kendall Park, NJ (US); Lyle P Kinne, Morrisville, PA (US); Amy Wrzesinski, Somerset, NJ (US); Paul Allenza, Flemington, NJ (US)

Correspondence Address: PATENT ADMINISTRATOR;FMC CORPORATION, 1735 MARKET STREET, PHILADELPHIA, PA, 19103, US

(21) Appl. No.: 10/512,376

(22) PCT Filed: Apr. 30, 2003
(86) PCT No.: PCT/US03/13170

§ 371 Date: Jun. 30, 2005

Related U.S. Application Data
(60) Provisional application No. 60/377,089, filed on May 1, 2002.

(51) Int. Cl. G01N 33/567

(52) U.S. Cl. 435/7.2

(57) ABSTRACT

The present invention discloses a human embryo kidney cell or cell line transfected with a GABA-gated chloride channel derived from an insect of the order of Lepidoptera, Diptera, Coleoptera, Homoptera, Acarina, Thysanaptera, Heteroptera, Hymenoptera or Isoptera and its use in a one-pot, high-throughput method for the measurement of the change in membrane potential in a cell or cell line. The method is particularly effective in measuring changes in membrane potential resulting from a compound acting on a GABA-gated chloride channel derived from a tobacco budworm.

This is a nonprovisional of provisional application Ser. No. 60/377,089, filed on May 1, 2002.

### FIELD OF THE INVENTION

The present invention relates generally to the field of ion channel activity. In particular, the invention relates to measuring the potential pesticidal activity of a compound by measuring membrane potential levels in cells or cell lines following chemical treatment, and more particularly it pertains to measuring the membrane potential in cells or cell lines transfected with a GABA-gated chloride channel following chemical treatment.

### BACKGROUND OF THE INVENTION

Gamma amino-n-butyric acid (“GABA”) gated chloride channels, which are receptors for GABA, play important roles in inhibiting synaptic transmission in both vertebrate and invertebrate nervous systems. Many existing biologically active compounds, for example insecticides, target this ion channel. Many of these compounds were initially identified based on their ability to act on the GABA receptor. Compounds directed to this site have quick modes of action. As such, there is a desire to develop ways to target this channel as a means for identifying biologically active compounds, including insecticides.

Cells or cell lines expressing insect ion channel receptors, in particular insect GABA-gated chloride channels, have been reported in the literature, for example Tomalski a et al., U.S. Pat. No. 5,854,002; Lee et al., FEBS Lett., 335(3), pp. 315-318 (1993); Shotkoski et al., FEBS Lett., 380(3), pp. 257-262 (1996); Buckingham et al., Neuropharmacology, 35(9/10), pp. 1393-1401 (1996); Smith et al., J. Recept. Signal Transduction Res., 15(1-4), pp. 33-41 (1995); Millar et al., Proc. R. Soc. London, Ser B. 258(1353) pp. 307-14 (1994); Halling et a., U.S. Pat. No. 6,329,516 B1; and Shotkoski et al., Insect Mol. Biol. 3(4) pp. 283-7 (1994). However, most of these cell lines are difficult to grow, are less stable, may not be conducive for use in high-throughput methods for measuring membrane potential. In addition, most of these cell lines tend to involve insect cell lines expressing the insect GABA receptor which is usually derived from the order of Aedes, Spdoptera, Trichoplusia, or Drosophila, resistant to dieldrine, or is a ligand-gated chloride channel homologue 3, rather than human embryo kidney (“HEK”) cell lines which express an insect GABA receptor derived from the order of Lepidoptera, Diptera, Coleoptera, Homoptera, Acarina, Thysanaptera, Heteroptera, Hymenoptera or Isoptera. As a result, there is a need for cells or cell lines that are easy to grow, are stable, conducive for use in high-throughput methods for measuring changes in membrane potential, and involve human embryo kidney cell lines rather than insect cell lines that express the insect GABA receptor.

Methods for measuring the ability of a compound to act on an insect GABA receptor are known to one skilled in the art. For example, one of ordinary skill in the art would know that electrophysiology measurements in oocytes expressing a functional channel can be used to test for insecticidal activity. Also, see Kellerman et al., WO 01/49848, Halling et al., U.S. Pat. No. 6,329,516 B1, and Tomalski et al., U.S. Pat. No. 5,854,002. However, most of these methods tend to involve cell lines that are difficult to grow, are less stable, and incorporate insect cell lines expressing the insect GABA receptor which is usually derived from the order of Aedes, Spdoptera, Trichoplusia, or Drosophila, resistant to dieldrine, or is a ligand-gated chloride channel homologue 3. These methods also tend not to be conducive for use in high-throughput methods given that they tend to incorporate a radioisotope or a ligand radiolabeled with a detectable isotope, which require special handling and can be expensive; measure membrane potential by electrophysiological methods rather than through fluorescence; use multiple reaction vessels; and be time-intensive limiting the number of measurements that can be carried out in a given time period. As such, these methods preclude a high-throughput method for measuring changes in membrane potential in a HEK cell or cell line transfected with a GABA-gated chloride channel derived from insects of the order of Lepidoptera, Diptera, Coleoptera, Homoptera, Acarina, Thysanaptera, Heteroptera, Hymenoptera or Isoptera that uses fluorescence. In addition, high-throughput methods reported in the literature, for example Molecular Devices Corporation's commercially available FLIPR® Membrane Potential Assay Kit (available from Molecular Devices, Sunnyvale Calif.) are not readily adaptable to all circumstances; tend to be less sensitive, not as reproducible. As a result, there is a need for an inexpensive, high-throughput method for measuring changes in membrane potential via fluorescence in a HEK cell or cell line expressing a GABA receptor gene.

### SUMMARY OF THE INVENTION

One embodiment of the present invention describes a cell or cell line useful in measuring a membrane potential change in a testing medium. The present invention comprises a HEK cell or cell line containing a GABA-gated chloride channel derived from an insect, preferably insects of the order of Lepidoptera, Diptera, Coleoptera, Homoptera, Acarina, Thysanaptera, Heteroptera, Hymenoptera or Isoptera.

Another embodiment of the present invention describes a cell or cell line comprising a HEK cell or cell line transfected with a tobacco budworm GABA-gated chloride channel, wherein the tobacco budworm GABA-gated chloride channel has the nucleotide sequence described in SEQ. ID NO: 4 or the amino acid sequence described in SEQ. ID NO: 5.

Yet another embodiment of the present invention describes a high-throughput method for measuring membrane potential. The present invention measures changes in fluorescence in response to the addition of a test compound. The present invention is particularly effective in measuring changes in fluorescence in the cells or cell lines described above.

In yet another embodiment of the present invention, a high-throughput method of identifying compounds that decrease fluorescence by comparing test compounds to a test medium alone or to the test medium following chemical treatment with compounds that decrease fluorescence is disclosed. This method can be useful in identify compounds suspected of exhibiting insecticidal activity.

In still yet another embodiment of the present invention, a high-throughput method of identifying compounds with insecticidal activity through the decrease in fluorescence in the cells or cell lines described above is disclosed.

The present invention is a high-throughput method that is less complex, more cost effective, and comparable in sensitivity to those disclosed in the art.

### DEFINITIONS

The term “ambient temperature” as utilized herein shall mean any suitable temperature found in a laboratory or other working quarter, and is generally not below about 15° C. nor above about 30° C.

The term “testing vessel” as utilized herein shall mean any device, such as a petri-dish, a microtiter plate, a test-tube, or beaker, which may be utilized to perform an assay, a reaction, a method, an experiment, or other procedure.

As used herein, the term “membrane potential indicator” shall mean any substance, such as a fluorescent dye, that is capable of indicating membrane potential change via fluorescence.

### BRIEF DESCRIPTION OF THE DRAWINGS

FIG. 1 shows the level of fluorescence of a human embryo kidney cell line transfected with a nucleotide sequence (SEQ. ID NO: 4) of a tobacco budworm following the standard protocol for the FLIPR® Membrane Potential Assay Kit.

FIG. 2 shows the level of fluorescence of a human embryo kidney cell line transfected with a nucleotide sequence (SEQ. ID NO: 4) of a tobacco budworm utilizing the present invention.

### DETAILED DESCRIPTION OF THE INVENTION

One embodiment of the present invention involves a cell or cell line comprising a human embryo kidney (“HEK”) cell or cell line transfected with a GABA-gated chloride channel derived from an insect. Preferably, the insect is of the order of Lepidoptera, Diptera, Coleoptera, Homoptera, Acarina, Thysanaptera, Heteroptera, Hymenoptera or Isoptera, more preferably of the order Lepidoptera or Homoptera, even more preferably of the order of Lepidoptera. Examples of insects that can be used in the present invention are European corn borers, cutworms, armyworms, tobacco budworms, seed maggots, mosquitoes, nuisance flies, beetles, rootworms, leafhoppers, aphids, mites, whiteflies, scales, stink bugs, thrips, wasps, ants, fruit worms, cabbage worms, moths, loppers, Lygus bugs, weevils, midges, leafminers, leafbeetles, or termites. Preferred insects are tobacco budworms and aphids. Particularly preferred insects are tobacco budworms. Examples of TBW GABA-gated chloride channels that can be used in the present invention are the nucleotide sequences described in SEQ. ID NO: 1, SEQ. ID NO: 4, or SEQ. ID NO: 7, preferably SEQ ID NO: 4, or the amino acid sequences described in SEQ. ID NO: 2, SEQ. ID NO: 5, or SEQ. ID NO: 8, preferably SEQ. ID NO: 5, as set forth herein and disclosed in U.S. Pat. No. 6,329,516 B1.

Another embodiment of the present invention describes a HEK cell or cell line transfected with a TBW GABA-gated chloride channel, wherein the tobacco budworm GABA-gated chloride channel has the nucleotide sequence of SEQ. ID NO: 4 or the amino acid sequence of SEQ. ID NO: 5, as set forth herein and disclosed in U.S. Pat. No. 6,329,516 B1.

Yet another embodiment of the present invention involves a method for measuring changes of membrane potential in the cell(s) or cell line(s) described above following chemical treatment, the method comprising:
(a) contacting a fixed amount of a membrane potential indicator with the test medium in a testing vessel;
(b) maintaining the membrane potential indicator in contact with the test medium in the testing vessel for a time sufficient to allow the membrane potential indicator to interact with the test medium;
(c) adding a test compound to the testing vessel;
(d) adding a fixed amount of GABA, wherein both the GABA and test compound desire to act on the GABA-gated chloride channel in the test medium; and
(e) measuring the level of fluorescence of the test medium, wherein the level of fluorescence is inversely proportional to the amount of the test compound acting on the GABA-gated chloride channel in said testing medium.

As set forth above, insects of the order of Lepidoptera, Diptera, Coleoptera, Homoptera, Acarina, Thysanaptera, Heteroptera, Hymenoptera or Isoptera, preferably, of the order of Lepidoptera or Homoptera, more preferably Lepidoptera, may be used in this embodiment. As set forth above, examples of insects that are useful include, but are not limited to, European corn borers, cutworms, armyworms, tobacco budworms, seed maggots, mosquitoes, nuisance flies, beetles, rootworms, leafhoppers, aphids, mites, whiteflies, scales, stink bugs, thrips, wasps, ants, fruit worms, cabbage worms, moths, loppers, Lygus bugs, weevils, midges, leafminers, leafbeetles, or termites. Preferred insects are tobacco budworms and aphids. Particularly preferred insects are tobacco budworms. As set forth above, the GABA-gated chloride channel may have the nucleotide sequences of SEQ. ID NO: 1, SEQ. ID NO: 4, or SEQ. ID NO: 7, preferably SEQ. ID NO: 4, or the amino acid sequences of SEQ. ID NO: 2, SEQ. ID NO: 5 or SEQ. ID NO: 8, preferably SEQ. ID NO: 5.

In preferred embodiments of the present invention, the GABA-gated chloride channel is a TBW GABA-gated chloride channel, wherein the TBW GABA-gated chloride channel has the nucleotide sequence of SEQ. ID NO: 4 or the amino acid sequence of SEQ. ID NO: 5.

Preferably, the test medium is a cell line transfected with a GABA-gated chloride channel derived from an insect. More preferably, the test medium is an HEK cell line transfected with a TBW GABA-gated chloride channel. Even more preferably, the test medium is an HEK cell line transfected with a TBW GABA-gated chloride channel wherein the TBW GABA-gated chloride channel has the nucleotide sequence of SEQ. ID NO: 4 or the amino acid sequence of SEQ. ID NO: 5.

Examples of membrane potential indicators that may be used in the present invention include, but are not limited to, fluorescent dyes, such as styryl dyes, cyanines, rhodamine probes, and oxonols.

The test compound may be contacted neat or as a solution in a solvent. Preferably, the test compound is contacted as a solution. As used herein the term “neat” refers to the unmixed or straight technical material along with any impurities contained therein. Examples of solvents which may be used in the present invention are saline solutions, for example potassium, sodium, or magnesium saline solutions, a tissue culture media, buffers, for example acidic, basic, or neutral buffers, water, an acid, a ketone, an alcohol, a sulfoxide, or mixtures thereof. Preferably, the test medium is added as a solution in a solvent and is comprised of a layer of living cells that grow and attach to the testing vessel. The solvents set forth above may also be used in connection with the test medium.

The time sufficient to allow the membrane potential indicator to interact with the test medium is preferably in the range of three to about five hours, more preferably 3.5 to 4.5 hours, at ambient temperature.

The level of fluorescence of the test medium, which is inversely proportional to the amount of the test compound acting on the GABA-gated chloride channel in said testing medium, can be measured by methods known to one skilled in the art, such as fluorometric methods, using equipment know in the art, such as a FLIPR³⁸⁴® Fluorometric Imaging Plate Reader system (available from Molecular Devices Corp.).

In another embodiment of the present invention, a method of identifying a compound which decreases the amount of fluorescence generated by a test medium is disclosed. The method comprises performing a trial utilizing the method disclosed above and then comparing the results form the trial to results produced from either:
(a) a negative control in which no compound is contacted with the testing medium;
(b) a positive control using a positive control compound as the test compound, wherein the positive control compound is a compound that decreases the fluorescence of the testing medium; or
(c) both a positive control and a negative control; wherein the fluorescence of the testing medium is less than the fluorescence that appears in the testing medium in the negative control and the fluorescence of the testing medium is more than or equal to the fluorescence that appears in the testing medium in the positive control is indicative of a test compound which can decrease the fluorescence that appears in a testing medium.

Preferably, the results from the trial are compared to results produced from both a positive control and a negative control; wherein the fluorescence of the testing medium is less than the fluorescence that appears in the testing medium in the negative control and the fluorescence of the testing medium is more than or equal to the fluorescence that appears in the testing medium in the positive control is indicative of a test compound which can decrease the fluorescence that appears in a testing medium.

Examples of positive control compounds that can be used in the present invention are fipronil, endosulfan, dieldrin and picrotoxin. A preferred and economical positive control compound is fipronil.

The method is particularly useful identifying compounds that exhibit insecticidal activity. The testing vessels, orders of insects, insects, GABA-gated chloride channels, nucleotide and amino acid sequences, and membrane potential indicators, including, but not limited to, the preferred testing vessels, orders of insects, insects, GABA-gated chloride channels, nucleotide and amino acid sequences, and membrane potential indicators, disclosed above can also be used in this embodiment.

As set forth above, the time sufficient to allow the membrane potential indicator to interact with the test medium is preferably in the range of three to five hours, more preferably 3.5 to 4.5, hours at ambient temperature. The methods of measuring level of fluorescence of the test medium set forth above can also be used in this embodiment.

In another embodiment of the present invention, a method of identifying a compound with insecticidal activity is disclosed. The method comprising:

i) performing a trial comprising the steps of:
(a) contacting a fluorescent dye with a human embryo kidney cell or cell line transfected with a tobacco budworm GABA-gated chloride channel having a nucleotide sequence of SEQ. ID NO: 4 in a microtiter plate;
(b) maintaining the fluorescent dye in contact with the human embryo kidney cell or cell line transfected with a tobacco budworm GABA-gated chloride channel having an amino acid of SEQ. ID NO: 4 in the microtiter plate for a time sufficient to allow the fluorescent dye to interact with the human embryo kidney cell or cell line transfected with a tobacco budworm GABA-gated chloride channel having a nucleotide sequence of SEQ. ID NO: 4;
(c) adding a test compound to the microtiter plate;
(d) adding a fixed amount of GABA to the microtiter plate, wherein both the GABA and test compound desire to act on the tobacco budworm GABA-gated chloride channel having a nucleotide sequence of SEQ. ID NO: 4 transfected into the human embryo kidney cell or cell line;
(e) measuring the level of fluorescence of the human embryo kidney cell or cell line transfected with a tobacco budworm GABA-gated chloride channel having a nucleotide sequence of SEQ. ID NO: 4, wherein the level of fluorescence is inversely proportional to the amount of the test compound acting on the tobacco budworm GABA-gated chloride channel having a nucleotide sequence of SEQ. ID NO: 4 transfected into the human embryo kidney cell or cell line; and

ii) comparing the results from the trial to results produced from either:
(a) a negative control in which no compound is contacted with the human embryo kidney cell or cell line transfected with a tobacco budworm GABA-gated chloride channel having a nucleotide sequence of SEQ. ID NO: 4;
(b) a positive control using a positive control compound as the test compound, wherein the positive control compound is a compound that decreases the fluorescence of the human embryo kidney cell or cell line transfected with a tobacco budworm GABA-gated chloride channel having a nucleotide sequence of SEQ. ID NO: 4; or
(c) both a positive and a negative control; wherein the amount of fluorescence in the human embryo kidney cell or cell line transfected with a tobacco budworm GABA-gated chloride channel having a nucleotide sequence of SEQ. ID NO: 4 is less than the fluorescence that appears in the human embryo kidney cell or cell line transfected with a tobacco budworm GABA-gated chloride channel having a nucleotide sequence of SEQ. ID NO: 4 in the negative control and the fluorescence of the human embryo kidney cell or cell line transfected with a tobacco budworm GABA-gated chloride channel having a nucleotide sequence of SEQ. ID NO: 4 is more than or equal to the fluorescence that appears in the human embryo kidney cell or cell line transfected with a tobacco budworm GABA-gated chloride channel having a nucleotide sequence of SEQ. ID NO: 4 in the positive control is indicative of a test compound which can decrease the fluorescence that appears in a testing medium.

The positive control compounds disclosed above can also be used in this embodiment. Preferably, the positive control compound is fipronil.

Preferably, the time sufficient to allow the dye to interact with the human embryo kidney cell or cell line transfected with a tobacco budworm GABA-gated chloride channel having a nucleotide sequence of SEQ. ID NO: 4 is in the range of three to four hours at ambient temperature. Similar to the methods disclosed above, the methods of measuring level of fluorescence of the test medium set forth above can also be used in this embodiment.

The level of fluorescence of the human embryo kidney cell or cell line transfected with a tobacco budworm GABA-gated chloride channel having a nucleotide sequence of SEQ. ID NO: 4 may be measured immediately or over a period of time at set intervals. For example, the level of fluorescence of the human embryo kidney cell or cell line transfected with a tobacco budworm GABA-gated chloride channel having a nucleotide sequence of SEQ. ID NO: 4 may be measured continuously at intervals of one to ten seconds over a specific period of time. Similar to the methods disclosed above, the methods of and equipment used for measuring the level of fluorescence of the test medium set forth above can also be used in this embodiment.

The present invention provides an improvement over other methods disclosed in the art in that it is a high-throughput method for measuring a changes in membrane potential of human embryo kidney cell or cell line transfected with a GABA-gated chloride channel derived from an insect as well as a means of identifying compounds with insecticidal activity which is less complex, more cost effective, yet comparable in sensitivity to those disclosed in the art.

The present invention is now described in more detail by reference to the following examples, but it should be understood that the invention is not construed as to be limited thereto.

### EXAMPLE 1

This example illustrates the construction of the mammalian expression vector for a tobacco budworm (“TBW”) GABA-A receptor.

The gene for TBW GABA-A receptor (TBW-a3, SEQ. ID. NO. 4, as disclosed in U.S. Pat. No. 6,329,516 B1, which is incorporated herein by reference) was in a plasmid vector pMT/V5-His A (“pmtALA1”, available from Invitrogen Corporation, Carlsbad, Calif.). In a test tube, pmtALA1 was transformed into methylation deficient (dam) bacterial strain, DM1 competent cells (available from Life Technologies Inc., Rockville, Md.) by methods know to one of ordinary skill in the art. The bacteria were grown at 37° C. for about sixteen hours. After this time, the pmtALA1 plasmid DNA containing TBW-a3 was isolated using a Qiagen Plasmid Mini Kit (available from Qiagen Inc., Valencia, Calif.), yielding isolated pmtALA1 plasmid DNA containing TBW-a3.

In a separate test tube, 17 μl (0.1 g/μl) of the above pmtALA1 plasmid was mixed with 2 μl of React 2 buffer (available from Life Technologies Inc.) and 1 μl of restriction enzyme Xba I (available from Life Technologies Inc.). The reaction mixture was incubated at 37° C. for one hour. At the conclusion of this period, the reaction mixture was separated on 1% agarose gel and the bands of linearized TBW-a3 DNA were excised. The excised TBW-a3 DNA was then purified using a QIAquick Gel Extraction Kit (available from Qiagen Inc.), yielding TBW-a3 DNA with Xba I sites on both ends.

In another separate test tube, 2 μl (1 ug/μl) of pcDNA3.1 (+) (DNA containing a human cytomegalovirus immediate early (CMV) promoter, available from Invitrogen Corporation) was mixed with 2 μl of React 2 buffer, 1 μl of Xba I, and 15 μl of water. The reaction mixture was incubated at 37° C. for 1 hour. At the conclusion of this period, the reaction mixture was separated on 1% agarose gel and the bands of linearized pcDNA3.1 (+) were excised. The linearized pcDNA3.1 (+) was purified using a QIAquick Gel Extraction Kit. Upon completion of purification, the linearized pcDNA3.1 (+) was dephosphorylated by mixing 1 μl (0.7 μg/μl) of the linearized pcDNA3.1 (+) with 4 μl of 10× shrimp alkaline phosphatase (“SAP”) buffer (available from Life Technologies Inc.), 15 μl of water and 20 μl of SAP (available from Life Technologies Inc.). The resulting mixture was incubated at 37° C. for 25 minutes, and then heat-inactivated by incubation at 65° C. for 20 minutes, yielding dephosphorylated plasmid pcDNA3.1 (+).

In yet another test tube, 1 μl of the above dephosphorylated plasmid pcDNA3.1 (+) was reacted with 6 μl (25 ng/μl) of the TBW-a3 DNA with Xba I sites on both ends, 2 μl of 5× ligase buffer (available from GIBCO-BRL, Hampstead, N.Y.), and 1 μl of T4 DNA ligase (available from GIBCO-BRL). The resulting mixture was incubated at 16° C. for about sixteen hours. At the conclusion of this period, 1 μl of the resulting mixture was diluted 10 fold in water, and a 1 μl aliquot was transformed into top 10 chemical competent bacteria by methods know to one of ordinary skill in the art. The transformed bacteria were then replicated on an agar plate containing LB media. Acceptable clones were selected and grown by methods know to one of ordinary skill in the art. The plasmid DNA containing TBW-a3 was isolated using a Qiagen Plasmid Mini Kit, yielding the plasmid pcDNA3.1(+) expressing the TBW-a3 GABA-A receptor, hereinafter referred to as “CMV-GABA-A”. The orientation of the CMV-GABA-A was determined by restriction digestion with Apa I.

### EXAMPLE 2

This example illustrates the generation of stable HEK cells constitutively expressing a TBW GABA-A receptor (hereinafter referred to as “HEK a-3 cells”).

A T75 flask of HEK cells (“HEK293TSA-O cells” available from Cell and Molecular Technologies, Phillipsburg, N.J.) was co-transfected with 2-5 μg of the CMV-GABA-A and 2-5 ug of a mammalian expression vector containing a puromycin resistant gene (“pPur Vector”, available from BD Biosciences Clontech, Palo Alto, Calif.) by using a LipofectAMINE PLUS™ Reagent package (available from Life Technologies Inc.) according to manufacturer's instructions. The transfected cells were selected for stable expression by growth in Dulbecco's Modified Eagle Medium (available from ATCC, Manassas, Va.) containing 4 μg/ml of puromycin (available from Clontech Laboratories Inc.) via methods know to one of ordinary skill in the art. A total of 48 resistant clones were grown in Dulbecco's Modified Eagle Medium containing 4 μg/ml of puromycin, as described above, and then frozen in two separate vials using liquid nitrogen. In parallel, total RNA was prepared from each resistant clone using TRIZOL reagent according to the manufacturer's instruction (Life Technologies, Rockville, Md.), and 1 μg of each preparation was spotted onto a Nytran filter (available from Shleicher and Scheull, Keene, N.H.). The filters were hybridized with a radioactive probe corresponding to the open reading frame of GABA-A gene TBW-a3 gene and washed with an aqueous sodium chloride/sodium citrate/sodium dodecyl sulfate solution. For the generation of the radioactive probe, linearized TBW-a3 cDNA was labeled using the T7 random prime labeling kit (Worthington Biochemical Corporation, Lakewood, N.J.) according to the manufacturer's instruction. The aqueous sodium chloride/sodium citrate/sodium dodecyl sulfate solution was prepared by dissolving 2.19 grams of sodium chloride, 1.10 grams of sodium citrate and 1 gram of sodium dodecyl sulfate in 800 ml of water; adjusting pH to 7.0 and then adjusting the volume to one (1) liter by adding water. After washing, the filter was wrapped in saran wrap, placed against a Kodak film with an intensifying screen on the other side of the film, and placed in an exposure cassette. The exposure cassette was stored at −80° C. for about sixteen hours. After this time, the film was developed and 25 clones were identified as having detectable levels of the GABA-A mRNA expression. These 25 clones were then grown in Dulbecco's Modified Eagle Medium containing 4 μg/ml of puromycin, as described above, yielding 25 clones of HEK a-3 cells that may be used in the assay to measure membrane potential.

### EXAMPLE 3

This example illustrates the measurement of fluorescence in HEK a-3 cells in the manner described in the protocol for the FLIPR® Membrane Potential Assay Kit.

The following solutions were prepared prior to or on the same day the experiment was to be carried out:
Solution A: To about one liter of deionized water was added 8.0 grams of sodium chloride, 0.395 gram of potassium chloride, 0.294 gram of calcium chloride, 0.203 gram of magnesium chloride, 4.5 grams of glucose, and 2.38 grams of [4-(2-hydroxyethyl)-1-piperazineethanesulfonic acid (“HEPES”) (all available from Aldrich Chemical Company, Milwaukee, Wis.). The resulting solution was stirred until dissolution was complete and then the pH adjusted to 7.3 to 7.4 with an aqueous 1 M sodium hydroxide solution.
Solution B: To one vile of the dye (“FLIPR® Membrane Potential Assay Reagent, Component A”) contained in the FLIPR® Membrane Potential Assay Kit was added about 10 ml of Solution A. The resulting mixture was mixed by repeated pippetting until the content of the vile was completely dissolved and used immediately or stored at 20° C. for future use.

The following solution was prepared on the same day the experiment was to be carried out:
Solution C: To solution B was added about 90 mL of Solution A. The resulting mixture was mixed until dissolution was complete.
Solution D: GABA (available from Sigma Chemical Co., St. Louis, Mo.), 30 grams, was taken up in 30 ml of Solution A. The resulting solution was mixed until dissolution was completion, yielding a 10 M GABA solution.
Solution E: To 1.2 ml of Solution D was added 38.8 ml of Solution A. The resulting mixture was mixed by repeated pippetting until dissolution was complete, yielding a 300 μM GABA solution.

One day before of the experiment, HEK a-3 cells were seeded into two wells of a poly-D-lysine coated blackwall 96 well microplate with clear bottom at a cell density of about 100,000 cells per well. Upon completion of addition, the cells were incubated at 37° C. in a cell culture incubator for twenty-four hours, for use below.

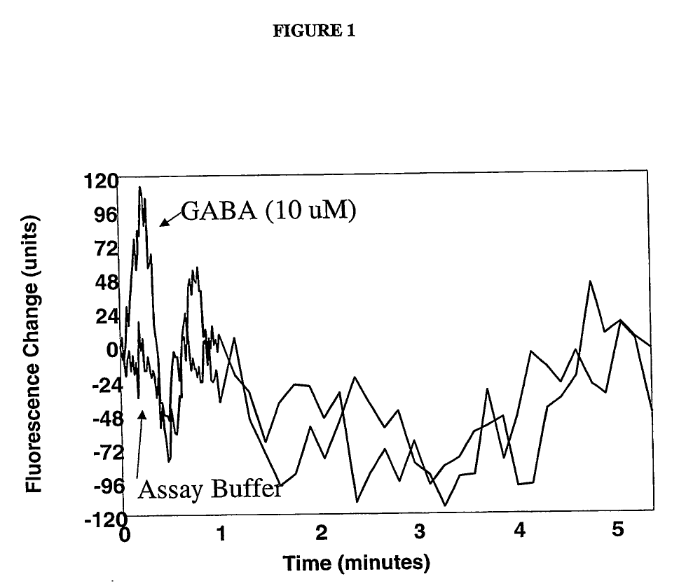

In carrying out the first step, on the day of the experiment, the 96 well microplate containing the HEK a-3 cells was removed from the incubator and 100 μl of Solution C was added to the two wells of the 96 well microplate. Upon completion of addition, the microplate was incubated in a cell culture incubator at 37° C. for 0.5 to one hour. After this time, the microplate was placed onto the FLIPR³⁸⁴® Fluorometric Imaging Plate Reader system in which the laser intensity was set at a suitable level to obtain a basal value of approximately 10,000 fluorescence units, fluorescent readings were obtained every one second for the first minute and then every six seconds thereafter, and fluorescence measurements were captured by a cooled CCD camera. After ten seconds from the start of the fluorescence measurements, 25 μl of Solution E was added at a rate of 25 μl/second by the system to one of the two wells of the 96 well microplate. Upon completion of addition, the level of fluorescence of the HEK a-3 cells was determined using the FLIPR³⁸⁴® Fluorometric Imaging Plate Reader system. Please note that no change in fluorescence was observed when the experiment was run as described above and in the standard protocol for the FLIPR® Membrane Potential Assay Kit. See FIG. 1.

### EXAMPLE 4

This example illustrates the measurement of fluorescence in HEK a-3 cells when the HEK a-3 cells, which interact with a fluorescent dye, are allowed to stand at ambient temperature for an extended period of time.

On the same day the experiment is to be carried, Solutions A-E are prepared again in the manner described above.

One day before of the experiment, HEK a-3 cells were seeded into two wells of a poly-D-lysine coated blackwall 96 well microplate with clear bottom at a cell density of about 100,000 cells per well. Upon completion of addition, the cells were incubated at 37° C. in a cell culture incubator for twenty-four hours, for use below.

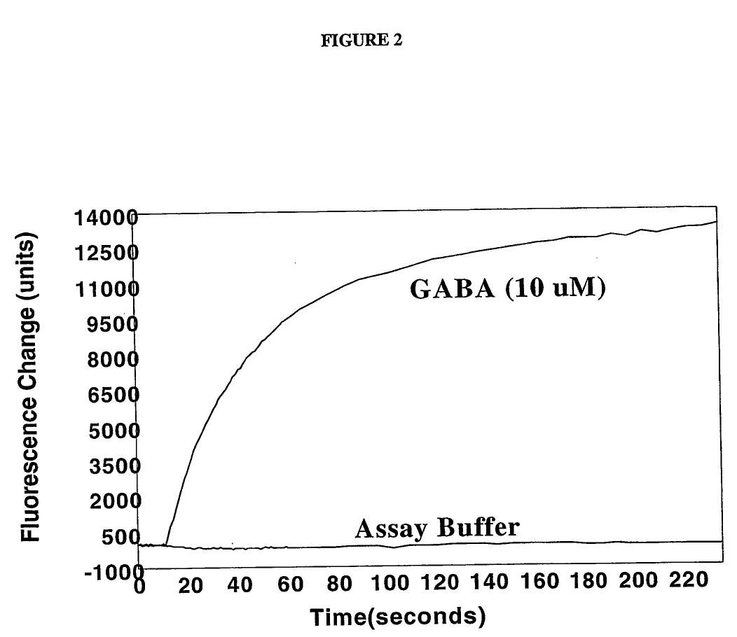

In carrying out the first step, on the day of the experiment, the 96 well microplate containing the HEK a-3 cells was removed from the incubator and 100 μl of Solution C was added to the two wells of the 96 well microplate. Upon completion of addition, the microplate was allowed to stand at ambient temperature of 3.5 to 4 hours. After this time, the microplate was placed onto the FLIPR³⁸⁴® Fluorometric Imaging Plate Reader system in which the laser intensity was set at a suitable level to obtain a basal value of approximately 10,000 fluorescence units, fluorescent readings were obtained every one second for the first minute and then every six seconds thereafter, and fluorescence measurements were captured by a cooled CCD camera. After ten seconds from the start of the fluorescence measurements, 25 μl of Solution E was added at a rate of 25 μl/second by the system to one of the two wells of the 96 well microplate. Upon completion of addition, the level of fluorescence of the HEK a-3 cells was determined using the FLIPR³⁸⁴® Fluorometric Imaging Plate Reader system. Please note that an increase in fluorescence in the HEK a-3 cells was observed when the cells were allowed to stand at ambient temperature for 3.5 to four hours. See FIG. 2.

### EXAMPLE 5

This example illustrates the measurement of fluorescence in HEK a-3 cells generated from fipronil using a fluorescent dye as the membrane potential indicator.

On the same day the experiment is to be carried, Solutions A-E are prepared again in the manner described above.

One day before of the experiment, HEK a-3 cells were seeded into each well of a poly-D-lysine coated blackwall 96 well microplate with clear bottom (available from Becton, Dickinson and Company, Franklin Lakes, N.J.) at a cell density of about 100,000 cells per well. Upon completion of addition, the cells were incubated at 37° C. in a cell culture incubator for twenty-four hours, for use below.

In carrying out the first step, on the day of the experiment, the 96 well microplate containing the HEK a-3 cells was removed from the incubator and 100 μl of Solution C were added to each well of the 96 well microplate. Upon completion of addition, the microplate was stored at ambient temperature for 3.5 to 4 hours. After this time, 22 μl of N,N-dimethylsulfoxide (“DMSO”, available from J. T. Baker Incorporated, final concentration—0.06%) was added to the wells designated as control wells and 22 μl of various dilutions, ranging in concentration from about 0.003 μM to 30 μM, of fipronil (available from Rhone-Poluenc, Inc. Research Triangle Park, N.C.) in DMSO were added to the wells designated for testing. Upon completion of addition, the microplate was allowed to stand at ambient temperature for 20 minutes to one hour. After this time, the microplates were placed onto the FLIPR³⁸⁴® Fluorometric Imaging Plate Reader system (available from Molecular Devices Corp.) in which the laser intensity was set at a suitable level to obtain a basal value of approximately 10,000 fluorescence units, fluorescent readings were obtained every one second for the first minute and then every six seconds thereafter, and fluorescence measurements were captured by a cooled CCD camera. After ten seconds from the start of the fluorescence measurements, 25 μl of Solution E were added by the system to the wells designated for testing. Upon completion of addition, the level of fluorescence of the HEK a-3 cells was determined using the FLIPR³⁸⁴® Fluorometric Imaging Plate Reader system. See Table 1 for results.

### EXAMPLE 6

This example illustrates the measurement of the membrane potential in HEK a-3 cells generated from endosulfan using a fluorescent dye as the membrane potential indicator.

This method was performed in the manner disclosed in Example 5 except that endosulfan was used rather than flipronil. See Table 1 for results.

As the results of Table 1 indicate, the present invention determined that all of the test compounds which are known to act on the GABA receptor decreased the level of fluorescence in the cell line even at low levels of concentration (see, for example, fipronil, which had 43% inhibition at the 0.01 micromolar (μM) concentration level) where as the controls and those compounds which are known not to act on the GABA receptor did not decrease the fluorescence at all (see, for example, bicuculine, which had 5% inhibition at the 10 micromolar (μM) concentration level). Thus, it will be seen that the present invention can be useful in predicting whether or not a prospective compound is likely to exhibit pesticidal activity because as set forth above the greater the decrease in fluorescence in the cell or cell line the more pesticidal activity a compound may possess.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

The present invention describes an improved method for measuring changes in membrane potential occurring in a human embryo kidney cell or cell line transfected with a GABA-gated chloride channel derived from an insect, preferably of the order lepidoptera, as well as an improved method for determining insecticidal activity. Said method is an improvement over those disclosed in the art in that it is a high-throughput method that is, more cost effective, has better reproducibility and is comparable in sensitivity to those disclosed in the art. Said method is also an improvement over the those disclosed in the art in that it utilizes membrane potential as measured by fluorescence rather than electrophysiological methods or binding ability through the use of a radioisotope or a ligand radiolabeled with a detectable isotope to indicate cellular response.

TABLE 1
Fluorescence and % inhibition of Pesticidally Active Compounds

| | Concentration | | |
|---|---|---|---|
| Compound | (μM) | Fluorescence⁴ | % Inhibition⁵ |
| Control¹ | — | 12509 | — |
| Fipronil² | 10 | 628 | 95 |
| | 3 | 1031 | 92 |
| | 1 | 1790 | 86 |
| | 0.3 | 2990 | 76 |
| | 0.1 | 4469 | 64 |
| | 0.03 | 6441 | 48 |
| | 0.01 | 8328 | 43 |
| | 0.003 | 11275 | 10 |
| Control¹ | — | 13833 | — |
| Endosulfan² | 10 | 993 | 93 |
| | 3 | 1615 | 88 |
| | 1 | 1903 | 86 |
| | 0.3 | 4426 | 68 |
| | 0.1 | 7098 | 48 |
| | 0.03 | 11074 | 20 |
| | 0.01 | 15198 | −9 |
| | 0.003 | 14343 | −3 |
| Control¹ | — | 13029 | — |
| Dieldrin² | 10 | 67 | 100 |
| | 3 | 900 | 93 |
| | 1 | 4184 | 68 |
| | 0.3 | 7279 | 44 |
| | 0.1 | 9924 | 34 |
| | 0.03 | 11492 | 12 |
| | 0.01 | 12339 | 5 |
| | 0.003 | 13284 | −1 |
| Control¹ | | 12140 | — |
| Bicuculine³ | 10 | 11543 | 5 |
| | 3 | ND | — |
| | 1 | ND | — |
| | 0.3 | ND | — |
| | 0.1 | ND | — |
| | 0.03 | ND | — |
| | 0.01 | ND | — |
| | 0.003 | ND | — |
| Control¹ | | 12140 | |
| Pentabarbitol³ | 10 | 11575 | 5 |
| | 3 | ND | — |
| | 1 | ND | — |
| | 0.3 | ND | — |
| | 0.1 | ND | — |
| | 0.03 | ND | — |
| | 0.01 | ND | — |
| | 0.003 | ND | — |

Notes:
¹DMSO
²Insecticides known to act at the GABA chloride channel
³Compounds known not to effect the insect GABA chloride channel
⁴Average of three (3) tests
⁵% Inhibition refers to the ability of the prospective compound to inhibit the GABA from acting on the available GABA receptors and is calculated as follows:
% Inhibition = [1 − (fluorescence of test compound/fluorescence of control)] × 100
ND indicates no significant difference from control

What is claimed:

1. A cell or cell line comprising a human embryo kidney cell or cell line transfected with a GABA-gated chloride channel derived from an insect.

2. The cell or cell line of claim 1, wherein the insect is of a order of Lepidoptera, Diptera, Coleoptera, Homoptera, Acarina, Thysanaptera, Heteroptera, Hymenoptera or Isoptera.

3. The cell or cell line of claim 1, wherein the GABA-gated chloride channel has a nucleotide sequence selected from the group consisting of: SEQ. ID NO: 1, SEQ. ID NO: 4, and SEQ. ID NO: 7.

4. The cell or cell line of claim 1, wherein the GABA-gated chloride channel has an amino acid sequence selected from the group consisting of: SEQ. ID NO: 2, SEQ. ID NO: 5, and SEQ. ID NO. 8.

5. A cell or cell line comprising a human embryo kidney cell or cell line transfected with a tobacco budworm GABA-gated chloride channel, wherein the tobacco budworm GABA-gated chloride channel has a nucleotide sequence of SEQ. ID NO: 4 or an amino acid sequence of SEQ. ID NO: 5.

6. A method for measuring levels of membrane potential in a test medium following chemical treatment, wherein the test medium comprises a cell or cell line comprising a human embryo kidney cell or cell line transfected with a GABA-gated chloride channel derived from an insect, the method comprising:
(a) contacting a fixed amount of a membrane potential indicator with the test medium in a testing vessel;
(b) maintaining the membrane potential indicator in contact with the test medium in the testing vessel for a time sufficient to allow the membrane potential indicator to interact with the test medium;
(c) adding a test compound to the testing vessel;
(d) adding a fixed amount of GABA, wherein both the GABA and test compound desire to act on the GABA-gated chloride channel in the test medium; and
(e) measuring the level of fluorescence of the test medium, wherein the level of fluorescence is inversely proportional to the amount of the test compound acting on the GABA-gated chloride channel in said test medium.

7. The method of claim 6, wherein the insect is of a order of Lepidoptera, Diptera, Coleoptera, Homoptera, Acarina, Thysanaptera, Heteroptera, Hymenoptera or Isoptera.

8. The method of claim 6, wherein the GABA-gated chloride channel has a nucleotide sequence selected from the group consisting of: SEQ. ID NO: 1, SEQ. ID NO: 4, and SEQ. ID NO: 7.

9. The method of claim 6, wherein the GABA-gated chloride channel has an amino acid sequence selected from the group consisting of: SEQ. ID NO: 2, SEQ. ID NO: 5, and SEQ. ID NO. 8.

10. The method of claim 6, wherein the GABA-gated chloride channel is a tobacco budworm GABA-gated chloride channel, wherein the tobacco budworm GABA-gated chloride channel has a nucleotide sequence of SEQ. ID NO: 4 or an amino acid sequence of SEQ. ID NO: 5.

11. The method of claim 6, wherein the membrane potential indicator is a fluorescent dye and the time to allow the membrane potential indicator to interact with the test medium is in the range of three to five hours at ambient temperature.

12. A method of identifying a compound which decreases the amount of fluorescence generated by a test medium, the method comprising:
i) performing a trial comprising the steps of:
(a) contacting a fixed amount of a membrane potential indicator with the test medium in a testing vessel, wherein the test medium comprises a cell or cell line comprising a human embryo kidney cell or cell line transfected with a GABA-gated chloride channel derived from an insect;
(b) maintaining the membrane potential indicator in contact with the test medium in the testing vessel for a time sufficient to allow the membrane potential indicator to interact with the test medium;
(c) adding a test compound to the testing vessel;
(d) adding a fixed amount of GABA, wherein both the GABA and test compound desire to act on the GABA-gated chloride channel in the test medium;
(e) measuring the level of fluorescence of the test medium, wherein the level of fluorescence is inversely proportional to the amount of the test compound acting on the GABA-gated chloride channel in said test medium; and
ii) comparing the results from the trial to results produced from either:
(a) a negative control in which no compound is contacted with the test medium;
(b) a positive control using a positive control compound as the test compound, wherein the positive control compound is a compound that decreases the fluorescence of the testing medium; or
(c) both a positive control and a negative control; wherein the fluorescence of the testing medium is less than the fluorescence that appears in the testing medium in the negative control and the fluorescence of the testing medium is more than or equal to the fluorescence that appears in the testing medium in the positive control is indicative of a test compound which can decrease the fluorescence that appears in a testing medium.

13. The method of claim 12, wherein said method is used to identify a compound that exhibits insecticidal activity.

14. The method of claim 12, wherein the insect is of a order of Lepidoptera, Diptera, Coleoptera, Homoptera, Acarina, Thysanaptera, Heteroptera, Hymenoptera or Isoptera.

15. The method of claim 12, wherein the GABA-gated chloride channel has a nucleotide sequence selected from the group consisting of: SEQ. ID NO: 1, SEQ. ID NO. 4 and SEQ ID. NO: 7.

16. The method of claim 12, wherein the GABA-gated chloride channel has an amino acid sequence of selected from the group consisting of: SEQ. ID NO: 2, SEQ. ID NO: 5, and SEQ. ID NO: 8.

17. The method of claim 12, wherein the GABA-gated chloride channel is a tobacco budworm GABA-gated chloride channel, wherein the tobacco budworm GABA-gated chloride channel has a nucleotide sequence of SEQ. ID NO: 4 or an amino acid sequence of SEQ. ID NO: 5.

18. The method of claim 12, wherein the membrane potential indicator is a fluorescent dye and the time to allow the membrane potential indicator to interact with the test medium is in the range of three to five hours at ambient temperature.

19. A method of identifying a compound with insecticidal activity, the method comprising:
i) performing a trial comprising the steps of:
(a) contacting a fluorescent dye with a human embryo kidney cell or cell line transfected with a tobacco budworm GABA-gated chloride channel having a nucleotide sequence of SEQ. ID NO: 4 in a microtiter plate;
(b) maintaining the fluorescent dye with the human embryo kidney cell or cell line transfected with a tobacco budworm GABA-gated chloride channel having a nucleotide sequence of SEQ. ID NO: 4 in the microtiter plate for a time sufficient to allow the fluorescent dye to interact with the human embryo kidney cell or cell line transfected with a tobacco budworm GABA-gated chloride channel having a nucleotide sequence of SEQ. ID NO: 4;
(c) adding a test compound to the microtiter plate;
(d) adding a fixed amount of GABA to the microtiter plate, wherein both the GABA and test compound desire to act on the tobacco budworm GABA-gated chloride channel having a nucleotide sequence of SEQ. ID NO: 4 transfected into the human embryo kidney cell or cell line;
(e) measuring the level of fluorescence of the human embryo kidney cell or cell line transfected with a tobacco budworm GABA-gated chloride channel having a nucleotide sequence of SEQ. ID NO: 4, wherein the level of fluorescence is inversely proportional to the amount of the test compound acting on the tobacco budworm GABA-gated chloride channel having a nucleotide sequence of SEQ. ID NO: 4 transfected into the human embryo kidney cell or cell line; and
ii) comparing the results from the trial to results produced from either:
(a) a negative control in which no compound is contacted with the human embryo kidney cell or cell line transfected with a tobacco budworm GABA-gated chloride channel having a nucleotide sequence of SEQ. ID NO: 4;
(b) a positive control using a positive control compound as the test compound, wherein the positive control compound is a compound that decreases the fluorescence of the human embryo kidney cell or cell line transfected with a tobacco budworm GABA-gated chloride channel having a nucleotide sequence of SEQ. ID NO: 4; or
(c) both a positive and a negative control; wherein the fluorescence of the human embryo kidney cell or cell line transfected with a tobacco budworm GABA-gated chloride channel having a nucleotide sequence of SEQ. ID NO: 4 is less than the fluorescence that appears in the human embryo kidney cell or cell line transfected with a tobacco budworm GABA-gated chloride channel having a nucleotide sequence of SEQ. ID NO: 4 in the negative control and the fluorescence of the human embryo kidney cell or cell line transfected with a tobacco budworm GABA-gated chloride channel having a nucleotide sequence of SEQ. ID NO: 4 is more than or equal to the fluorescence that appears in the human embryo kidney cell or cell line transfected with a tobacco budworm GABA-gated chloride channel having a nucleotide sequence of SEQ. ID NO: 4 in the positive control is indicative of a test compound which can decrease the fluorescence that appears in a testing medium.

20. The method of claim 19, wherein the time to allow the fluorescent dye to interact with the human embryo kidney cell or cell line transfected with a tobacco budworm GABA-gated chloride channel having a nucleotide sequence of SEQ. ID NO: 4 is in the range of three to four hours at ambient temperature.
